(19) United States
(12) Patent Application Publication
(10) Pub. No.: US 2010/0016210 A1
(43) Pub. Date: Jan. 21, 2010

(54) Sensotherapy

(76) Inventor: Michael Cherkassky, Fort Worth, TX (US)

Correspondence Address: Michael Cherkassky;Suite 418, 1550 W. Rosedale, Fort Worth, TX, 76104, US

(21) Appl. No.: 12/218,968

(22) Filed: Jul. 21, 2008

(51) Int. Cl. A61K 38/00; A61P 3/04

(52) U.S. Cl. 514/2

(57) ABSTRACT

The invention involves a preparation for the weight loss by means of appetite suppression and a method for administering the preparation to humans and other mammals.

A preparation consists of a variety of different spices, sugars, and salts as well as a proteins and alcohols.

### BACK GROUND

### Field of Invention

Overweight and obesity our major problems within the Western Community due to increased consumption and changes in nutritional value many humans and companion animals are suffering from overweight. Today obesity is one of the most serious health problems in the United States approximately 30% of the adults in the country suffer from obesity and at least 50% of the adults in the United States are overweight. The problem of obesity in United States and the most western countries has been steadily increasing over the last several decades. Obesity has caused or contributed to a marked increase in the occurrence of heart diseases, hypertension, diabetes, arthritis, and increased morbidity and mortality. There is also recent research which links obesity with different types of cancer particularly breast cancer. Obesity is a serious public health hazard. Being overweight reduces lifespan as well as quality of life.

There are many methods that suggest management of obesity and overweight. They include diets that exclude fats and high caloric elements, appetite suppressants, number of psychotherapeutic techniques, and variety of operative techniques. One of the most common methods is the use of the stimulants: amphetamine-like agents would surely act on the brain to reduce the sensation of hunger. Experience indicates that most of the appetite suppressants work for a short period of time “a few weeks or a few months” after that they lose most of their potency and patients start regaining weight.

There is also a serious problem with the maintenance of a desirable level of weight after it is achieved for a simple reason that appetite suppressants can not be continued indefinitely.

The reason most people become overweight is that they consume more nutrition “calories” that is required for them. The abandons of food and the fact that food is readily available as well that it is a relatively inexpensive in the developed countries resulted into inordinate consumption which is regulated not by the necessity but by the satisfaction of the plate. In other words we eat not to survive but for the taste, flavor and gratification. The degree of the satiety has changed and predicated not on the necessary nutritional requirements but on unphysiological “unnatural” drive to pleasure.

Complex mechanism which is triggered in mammals body in the process of food digestion is characterized by multiple inter dependent processes where different hormones with multiple functions influence different organs at the same time and by that allow the whole body function properly.

One of the necessary sensations which complete the mechanism of digestion is satiety. They and many unknown areas in our knowledge of the satiety but the number of hormones known to participate in the process of digestion also play a significant role in inducing a sense of the satiety.

There is a group called gastrointestinal peptides. They predominantly polypeptides produced in and secreted from specialized gut endocrine cells as well as nerves.

The production of gastrointestinal hormones increases when gut endocrine cells are stimulated by food, intraluminal pH, releasing factors, other transmitters or hormones. There are a number of fairly well known gastrointestinal hormones: Amylin, CCK, Gastrin, Secretin, Enterostatin, polypeptide Y 3-36.

All of these hormones play their specific role in digestion processes confined to the intestinal tract but they also participate in transmitting information to the brain enabling the brain to be well appraised of the quantity and quality of food being consumed and thus modulating and regulating the amount of food intake from meal to meal.

Information from the gastrointestinal tract and oropharynx is newly transmitted to the brain.

It has been proven that this hormone reaches tractus solitarius via hypothalamus and concentrating over there they induce satiety. Among them CCK, Amylin, and possibly Insulin.

Amylin and CCK also reduce gastric emptying and intestinal mobility and thus delaying the delivering of food to the intestines and contributing to the early sense of the satiety end as a result limiting overall quantity of food being consumed.

The above description illustrates that there's some mechanisms triggered by food consumption also play a role in limiting quantity of food being consumed during one particular meal.

It was postulated by the author of this patent that by using certain food ingredients with limited nutritional value but capable of stimulating intestinal tract it will be possible to reach early stage of the satiety.

There are a number of ingredients in food which would fit to the description: pepper, mustard, cinnamon, sugar, sugar substitutes, salt, alcohol, proteins, albumins and Cellulose.

### SUMMARY OF THE INVENTION

It is the object of the present invention to develop a composition of the above mentioned ingredients that can effectively act to control the weight of the mammal to which composition is administered. It is a further object of the present invention to develop weight control composition of food products that can act to control the weight of a mammal to which the composition is administered through each of suppressing appetite inducing early sense of the satiety. It is still further object of the present invention to develop a method of administering a composition of food products to control weight without notable side effect.

These and other objects partly are achieved by a method of administering a composition of pepper, mustard, sugar, salt, alcohol, proteins, albumins, cellulose, and a variety of other spices sugars and salts, to mammal preferably human in a manner effective to control the weight of the mammal.

In the method mention above, composition is preferably administered orally. This object is also achieved by weight control composition a different food ingredients containing at least some spices, sugars, salts, and proteins.

### DETAILED DESCRIPTION OF THE PREFERRED EMBODIMENT

The preferred embodiment of the invention involves the administration to mammals of a variety of specially combine food ingredients with limited nutritional value which singly or in combination capable of stimulating intestinal hormones which in its turn would influence brain specifically “tractus solitarius” and induce early satiety and thus drastically limiting quantity of food consumed at any particular meal, the same intestinal hormones also suppose to reduce gastric emptying resulting to the same goal as of limiting food consumption.

### Dosages and Preparations

The single oral dosage consists of 0.8 cc capsule with approximately 70% of cellulose, 20% of proteins, 0.1% of pepper, 0.1% of saccharin, 0.2% of mustard, 2% salt, 0.5% of alcohol the proportion of the ingredients could very as well as some other unions of similar made she could be included, initially the whole preparation is thoroughly mixed and evaporated in the microwave, obviously some other temperature treatments could be applied including a stove or a freezer.

The three types of preparations one is the basic one described above.

The second one, with the inclusion of 200 mg of caffeine which is designed to be used in the early part of the day.

The third one which has approximately 90 mg of caffeine which is used in the later part of the day. The reason for the caffeine inclusion is that during the weight loss processes people frequently feel exhausted and tired they need certain boost of energy.

It is interesting to note that the basic preparation, the one which does not have caffeine is the most convenient for the night time use for there is practically no appetite suppressants remedy available at present which is not a stimulant. As it was mentioned before with a few exceptions appetite suppressants are stimulants by nature and cannot be used before sleeping hours leading to a tremendous gap of unprotection to the patient.

Another interesting aspect of this new discovery is that non caffeine preparations could be used to enhance the appetite suppressants effect of the caffeine containing preparations which means that it is possible to extend appetite suppression too a much higher degree, then it is available at present.

Still another advantage is that a new discovery could be combined with any other commercially available appetite suppressants or for that matter anything which will be discovered by the signs in the future because that particular preparation has virtually no side effects.

In the still another embodiment the above principles could be applied utilizing a fact which recognizes a close connection between the tongue, oral cavity, oropharynx and intestines, when small tablet is placed in the mouth without being swallowed and that would allow to trigger similar chain of events resulting to early satiety. The tablet consists of the combination of different spices saccharin and salts.

The extension of the above idea could be an application of the paste consisting of spices sugar and salt on the selected parts of the tongue with simultaneous feeding of the small quantity of food products smeared with the said paste. The food products naturally would be presented by the ones patients crave most. Thus allowing the patient to eliminate or greatly reduce the dependence on the products they crave.

1. I claim a process for causing weight loss or avoidance of weight gain in mammals comprising of oral administration to said mammals is a composition of: condiments salts sweeteners alcohol proteins carbohydrates fats delivered in pharmaceutical dosage form such as capsules, tablets, troche whereby all of the above ingredients together effectively diminish appetite

2. The composition of claim 1 wherein alcohol is added to the above ingredients in the liquid form and then evaporated resulting into a powder.

3. The composition of claim 1 delivered in the form of a small tablet to be placed in the oral cavity to be sucked upon without a process of swallowing.

4. The composition of claim 1 delivered in the form of the paste to be applied on the designated parts of the tongue.
